(19) United States
(12) Patent Application Publication
(10) Pub. No.: US 2008/0302950 A1
(43) Pub. Date: Dec. 11, 2008

(54) System and Method for Performing Single Photon Emission Computed Tomography (Spect) with a Focal-Length Cone-Beam Collimation

(75) Inventors: Mi-Ae Park, Newton, MA (US); Stephen C. Moore, Farmingham, MA (US); Marie Foley Kijewski, Mamaroneck, NY (US)

Correspondence Address: DORSEY & WHITNEY LLP;INTELLECTUAL PROPERTY DEPARTMENT, 250 PARK AVENUE, NEW YORK, NY, 10177, US

(73) Assignee: The Brigham and Women's Hospital, Inc., Boston, MA (US)

(21) Appl. No.: 12/063,131

(22) PCT Filed: Aug. 11, 2006
(86) PCT No.: PCT/US06/31627

§ 371 Date: Jul. 8, 2008

Related U.S. Application Data
(60) Provisional application No. 60/707,734, filed on Aug. 11, 2005.

(51) Int. Cl. A61B 6/00; G02B 27/30

(52) U.S. Cl. 250/216; 359/641

(57) ABSTRACT

A system and method are provided for obtaining data that may be used to generate images of a brain or other bodily organ. The system can include a pair of detecting arrangements and a collimating arrangement associated with each detecting arrangement. A first collimating arrangement can include a cone-beam collimating arrangement having a focal point located within the brain or other organ being imaged. A second collimating arrangement can include a fan-beam collimating arrangement having a focal length selected such that the organ being imaged lies within its field of view to ensure data sufficiency. Cone-beam collimating arrangements having improved hole geometries can also be utilized to provide further increases in imaging sensitivity.

### CROSS-REFERENCE TO A RELATED APPLICATION

This application claims priority from U.S. patent application Ser. No. 60/707,734 filed on Aug. 11, 2005, the entire disclosure of which is incorporated herein by reference.

### FIELD OF THE INVENTION

The present invention relates to an apparatus for imaging biological tissue, and more particularly to an apparatus for imaging of a brain that can include a dual-head camera arrangement, a cone-beam collimating arrangement having a first focal length, and a fan-beam collimating having a second focal length which can be longer than the first focal length.

### BACKGROUND INFORMATION

Single Photon Emission Computed Tomography (“SPECT”) imaging techniques may use converging collimation to increase imaging sensitivity. These techniques can use fan-beam collimating arrangements capable of focusing transaxially, as described in, e.g., R. J. Jaszczak et al., “Single photon-emission computer-tomography using multi-slice fan beam collimating arrangements,” IEEE Trans. Nucl. Sci. 26, 610-618 (1979); C. B. Lim et al., “Performance analysis of 3 camera configurations for single photon-emission computer-tomography,” IEEE Trans. Nucl. Sci. 27, 559-568 (1980); and B. M. W. Tsui et al., “Design and clinical utility of a fan beam collimating arrangement for SPECT imaging of the head,” J. Nucl. Med. 27, 810-819 (1986). A cone-bean collimating arrangement that is capable of focusing both transaxially and axially may also be used. Such apparatus is described in R. J. Jaszczak et al., “Cone beam collimation for single photon-emission computer-tomography-Analysis, simulation, and image-reconstruction using filtered back projection,” Med. Phys. 13, 484-489 (1986). An astigmatic converging collimating arrangement as described, e.g., in E. G. Hawman and J. Hsieh, “An astigmatic collimating arrangement for high-sensitivity SPECT of the brain,” J. Nucl. Med. 27, 930 (1986) may also be used to achieve a higher sensitivity.

Converging collimating arrangements can also improve resolution through image magnification effects. Sensitivity and resolution gains of converging collimating arrangements can be increased by decreasing the focal length and placing the collimating arrangement closer to the head. The extent to which the focal length can be decreased, however, may be limited by a need to have a field of view large enough to encompass an entire brain, and the need to avoid shoulders. Techniques that may be used to address these limitations include tilting the collimating arrangement as described, for example, in R. J. Jaszczak et al., “SPECT using a specially designed cone beam collimating arrangement,” J. Nucl. Med. 29, 1398-1405 (1988) or using half-cone-beam collimating arrangements such as those described in J. Y. Li et al., “Half-cone beam collimation for triple-camera SPECT systems,” J. Nucl. Med. 37, 498-502 (1996). Another technique that may be used is to offset the focal points of a pair of cone-beam collimating arrangements axially as described, e.g., in C. Kamphuis and F. S. Beekman, “The use of offset cone-beam collimating arrangements in a dual head system for combined emission transmission brain SPECT: a feasibility study,” IEEE Trans. Nucl. Sci. 45, 1250-1254 (1998), and in D. S. Lalush, “Dual-planar circular-orbit cone-beam SPECT,” J. Nucl. Med. 39, 22 (1998).

SPECT tracers that may be specific for neurotransmifter systems can be utilized to improve the sensitivity of striatal imaging. SPECT agents which bind to dopamine transporter sites may be used, including 1-123 agents beta-CIT which are described, e.g., in M. Laruelle et al., “Graphical, kinetic, and equilibrium analyses of in-vivo [I-123] beta-CIT binding to dopamine transporters in healthy-human subjects,” J. Cereb. Blood Flow Metab. 14, 982-994 (1994). Other agents that may be used include altropane, which is described in A. J. Fischman et al., “Rapid detection of Parkinson's disease by SPECT with altropane: A selective ligand for dopamine transporters,” Synapse. 29, 128-141 (1998), or a Tc-99m agent, TRODAT, described in M. P. Kung et al., “[Tc-99m]TRODAT-1: A novel technetium-99m complex as a dopamine transporter imaging agent,” Eur. J. Nucl. Med. 24, 372-380 (1997). A dopamine receptor agent, IBZM, which is described in H. F. Kung et al., “In vitro and in vivo evaluation of [I-123]-EBZM: a potential CNS D-2 dopamine receptor imaging agent,” J. Nucl. Med. 30, 88-92 (1989), may also be used to improve imaging sensitivity.

Quantitative estimates of striatal activity concentrations and volumes may be clinically significant in several neurological diseases. Reductions in the size and activity concentration of striata have been reported in Parkinson disease as described, for example, in the Fischman publication, and in R. B. Innis et al., “Single-photon emission computer homographic imaging demonstrates loss of striatal dopamine transporters in Parkinson disease,” Proc. Natl. Acad. Sci. 90, 11965-11969 (1993); S. Asenbaum et al., “Imaging of dopamine transporters with Iodine-123-beta-CIT and SPECT in Parkinson's disease,” J. Nucl. Med. 38, 1-6 (1997); J. Booij et al., “[I-123]FP-CIT SPECT shows a pronounced decline of striatal dopamine transporter labeling in early and advanced Parkinson's disease,” J. Neur. Neuros. Psy. 62, 133-140 (1997); A. Winogrodzka et al., “[I-123] beta-CIT SPECT is a useful method for monitoring dopaminergic degeneration in early stage Parkinson's disease,” J. Neur. Neuros. Psy. 74, 294-298 (2003); A. Varrone et al., “[I-123]beta-CIT SPECT imaging demonstrates reduced density of striatal dopamine transporters in Parkinson's disease and multiple system atrophy,” Movement Disorder 16, 1023-1032 (2001); and T. Ishikawa et al., “Comparative nigrostriatal dopaminergic imaging with iodine-123-beta CIT-FP/SPECT and fluorine-18-FDOPA/PET,” J. Nucl. Med. 37, 1760-1765 (1996). Abnormalities of the dopaminergic system have also been reported in other movement disorders, including Huntington disease as described, e.g., in M. Ichise et al., “Iodine-123-IBZM Dopamine-De receptor and Techlietium-99m-HMPAO brain perdusion SPECT in the evaluation of patients with and subjects at risk for Huntingtons-disease,” J. Nucl. Med. 34, 1274-1281 (1993). Perfusion imaging of central structures is of interest in Alzheimer disease; reduced perfusion in the hippocampal complex and the cingulate may be related to changes in memory and executive function, respectively, as described in K. A. Johnson et al., “Preclinical prediction of Alzheimer's disease using SPECT,” Neurology. 50, 1563-1571 (1998). Furthermore, neuroimaging of dopamine function may enable an identification of a recently recognized subset of Alzheimer patients who may now be diagnosed post mortem by the presence of Lewy bodies in the brain, which is described in E. Donnemiller et al., “Brain perfusion scintigraphy with Tc-99m-HMPAO or Tc-99m-ECD and I-123-beta-CIT single-photon emission tomography in dementia of the Alzheimer-type and diffuse Lewy body disease,” Eur. J. Nucl. Med. 24, 320-325 (1997). Results of molecular genetic studies have indicated that attention deficit hyperactivity disorder (“ADHD”) may be associated with abnormalities in the dopaminergic system as described, for example, in S. V. Faraone and J. Biedennan, “Neurobiology of attention-deficit hyperactivity disorder,” Biol. Psych. 44, 951-958 (1996).

Certain information for altered dopamine function in ADHD has emerged from SPECT and PET studies such as that described in D. D. Dougherty et al., “Dopamine transporter density in patients with attention deficit hyperactivity disorder,” Lancet. 354, 2132-2133 (1999). Several of these studies have indicated an improvement after a treatment with methylphenidate, including studies described in K. H. Krause et al., “Increased striatal dopamine transporter in adult patients with attention deficit hyperactivity disorder: effects of methylphenidate as measured by single photon emission computed tomography,” Neurosci. Lett. 285, 107-110 (2000); S. Dresel et al., “Attention deficit hyperactivity disorder: binding of [(TC)-T-99m]TRODAT-1 to the dopamine transporter before and after methylphenidate treatment,” Eur. J. Nucl. Med. 27, 1518-1524 (2000); and N. D. Volkow et al., “Therapeutic doses of oral methylphenidate significantly increase extracellular dopamine in the human brain,” J. Neuroscience. 21, U1-U5 (2001).

In some of these clinical applications, it may be possible to estimate kinetic parameters from striatal activity curves which can be extracted from rapidly acquired image sequences. An improved sensitivity can be important for dynamic implementation, in which low sensitivity may not be compensated by increasing imaging time. An efficient estimation of kinetic parameters using nonlinear estimation techniques may not be possible when images are too noisy, as described in S. P. Mueller et al., “Estimation performance at low SNR: predictions of the Barankin bound,” in Medical Imaging: Physics of Medical Imaging. Richard L, van Metter, and Jacob B, Eds., Proc. SPIE 2432, 152-166 (1995) and in S. P. Mueller et al., “Chi-squared isocontours: predictors of task performance in nonlinear estimation tasks at low SNR,” in Medical Imaging. Ed. Hanson K M, Proc. SPIE, 3034, 176-187 (1997). Thus, an improved sensitivity, particularly near the center of the brain, may have a significant impact on the diagnosis and management of a number of serious neurological diseases.

It may be possible to reduce image noise over most of an imaging volume with little or no changes in injected activity or imaging time by using a centrally peaked collimating arrangement sensitivity function, e.g., detecting relatively more counts from the central (in the transaxial direction) portion of the projections. This technique is described, e.g., in M. F. Kijewski et al., “Nonuniform collimating arrangement sensitivity: Improved precision for quantitative SPECT,” J. Nucl. Med. 38, 151-156 (1997). An exemplary collimating arrangement using this technique has been designed and manufactured for a dedicated brain SPECT instrument with cylindrical geometry and is described in S. Genna et al., “Annular single-crystal emission tomography systems” in The fundamentals of PET and SPECT (Wernick M N and Asrsvold J N), Academic Press, 2004 (in press). However, it may not be possible or feasible to achieve a centrally-peaked sensitivity function and, consequently, a greatly improved count sensitivity from central brain structures using conventional dual- or triple-head SPECT systems.

SPECT imaging of deep brain structures may be compromised by a loss of photons arising from attenuation. A centrally peaked collimating arrangement sensitivity function can compensate for this phenomenon, which may increase sensitivity over most of the brain. For dual-head instruments, parallel-hole collimating arrangements generally may not provide a variable sensitivity without simultaneously degrading spatial resolution near the center of the brain.

Studies of SPECT tracers that can be specifically configured for neurotransmitter systems indicate a possible need for improving sensitivity of striatal imaging. However, photons emitted from the central region of the brain may be preferentially attenuated, leading to increased image noise. Geometrical sensitivity may be determined by a collimating arrangement, the first structure that an emitted photon can encounter after exiting a portion of the patient. Conventional collimating arrangements can include parallel-hole collimating arrangements and fan-beam and cone-beam collimating arrangements with long focal lengths (>40 cm). It may be difficult to increase sensitivity in the central brain regions sufficiently to overcome the effects of attenuation with such collimating arrangements which can be used with dual- or triple-head SPECT systems.

### OBJECTS AND SUMMARY OF THE INVENTION

One of the objects of the present invention is to overcome at least some of the deficiencies of the conventional systems and methods described above using exemplary embodiments of the present invention.

According to one exemplary embodiment of the present invention, a pair of collimating arrangements can be used with a further radiation detecting arrangement to increase sensitivity, e.g., in a center of the brain. This exemplary configuration may improve an estimation of an activity concentration of small structures at various locations in the brain of the patient. The exemplary collimating arrangements may include a cone-beam collimating arrangement to provide increased sensitivity and a fan-beam collimating arrangement to provide data sufficiency. It may be possible to determine projections of an ellipsoidal uniform background with, e.g., 0.9-cm-radius spherical lesions at several locations in the background. Such a calculation may provide an approximation for a signal-to-noise ratios (SNR_{CRB}) that can assist with an estimation of activity concentration within the spheres based on a Cramer-Rao lower bound on variance. It may also be possible to reconstruct, using an exemplary OS-EM procedure, images of this “phantom” configuration, as well as images of a Zubal brain phantom, to provide improved visual assessment and help ensure that it may be substantially free of artifacts.

According to one exemplary embodiment of the present invention, a cone-beam collimating arrangement may be used which has a focal point provided within the brain or other organ to be imaged, e.g., it may have a focal length of about 20 cm. A fan-beam collimating arrangement may also be used where the brain or other organ to be imaged lies within the field of view of the fan-beam collimating arrangement, e.g., it may be provided with a focal length of about 40 cm. These collimating arrangements (e.g., pairs) may yielded an increased SNR_{CRB} compared to a parallel-parallel pair used throughout the imaging volume. The factor by which SNR_{CRB} can be increased may range, e.g., from about 1.1 at an axially extreme location to about 3.5 at the center. The gain in SNR_{CRB} may be relatively insensitive to mismatches between the center of the brain and the center of the imaging volume. Artifact-free reconstructions of simulated data may be acquired using this pair. Thus, combining fan-beam and short-focusing cone-beam collimation as described herein can improve dual-head brain SPECT imaging, particularly for centrally located structures.

According to a further exemplary embodiment of the present invention, a cone-beam collimating arrangement may be mounted on a SPECT camera or other detector, and used together with a conventional fan-beam collimating arrangement provided on a second camera or detector to obtain data related to an image of the brain or organ of interest.

In further exemplary embodiments of the present invention, a pair of collimating arrangements can be provided that includes an ultra-short cone-beam (USCB) collimating arrangement having a focal length of about 20 cm, for which the focal point is inside the brain, and a fan-beam collimating arrangement having a focal length of about 40 cm. The gain in sensitivity for this combination compared to conventional parallel-beam collimation may range from about 1.5 at the periphery to about 10 at the center of the brain. For a sphere located at the center of the brain, the SNR_{CRB} for this collimating arrangement pair may be about 3.5 times greater than that of a parallel-parallel collimating arrangement pair. Artifact-free reconstructed images can be obtained using this collimating arrangement pair, and position mismatches between the center of the brain and the center of the scanner imaging volume of up to 14.4 mm may not significantly reduce the advantages of this collimating arrangement pair as compared with conventional parallel-beam collimating arrangements.

Another object of the present invention is provide various exemplary collimating arrangements which can be used with conventional dual-head SPECT instruments that are capable of increasing sensitivity of brain imaging. According to another exemplary embodiment of the present invention, a collimation system can be provided for dual-head SPECT cameras which can include a hybrid ultra-short focusing/slant-hole collimating arrangement that can provide increased central sensitivity, and a fan-beam collimating arrangement that can provide data sufficiency. This exemplary collimating arrangement can be referred to as a hybrid ultra-short cone-beam/slant-hole (“USCB/S”) collimating arrangement; and it may allow for a retention of sensitivity gains of a USCB collimating arrangement while eliminating the need for large hole angulations. The USCB/S collimating arrangement may also provide an improved spatial resolution as compared to parallel-hole collimation arising from magnification effects. The improved sensitivity, e.g., near the center of the brain, may provide significant improvements in diagnosis and management of a number of neurological diseases.

The USCB/S collimating arrangement can be optimized based on, e.g., a performance in activity estimation; focal length, hole size and/or septal thickness. For example, a collimating arrangement thickness can be determined for the two radionuclides most frequently attached to tracers used in brain imaging, ^{99m}Tc and ¹²³I. Sensitivity as a function of point-source position in a brain-sized ellipsoidal attenuator can be determined using analytic aperture functions and Monte Carlo (MC) simulations. A focal length of the USCB portion of this collimating arrangement may be selected based on these sensitivity profiles. The USCB/S collimating arrangement can be paired with a fan-beam collimating arrangement having a focal length of about 40 cm to ensure data adequacy. The collimating arrangement thickness, hole size, and/or septal thickness can be optimized for ¹²³Iand for ^{99m}Tc, for both the USCB/S collimating arrangement and the 40-cm-focal length fan-beam collimating arrangement. This exemplary optimization can be based on performance in estimation tasks using images reconstructed from MC-simulated data. The collimating arrangement pair can be selected for each radionuclide of interest based on performance in estimation of activity concentration of several spheres embedded in an ellipsoidal phantom. Because it may not be possible or desirable to purchase separate collimating arrangements for ¹²³I and ^{99m}Tc, it may also determine the performance of a ¹²³I collimating arrangement when it is used to image ^{99m}T signals. For example, most or all collimating arrangements can be evaluated on the basis of accuracy and precision of an estimation of activity concentration of several brain structures based on images reconstructed from simulated data that may be anatomically and physiologically realistic. Such data may be obtained, e.g., by an MC simulation using tracer distributions characteristic of ^{99m}Tc-HMPAO brain scans of normal subjects and patients with Alzheimer disease, or by ¹²³I-altropane brain scans of normal subjects and patients with Parkinson disease.

The higher sensitivity which can be provided by collimation systems in accordance with exemplary embodiments of the present invention may allow more precise estimates of striatal activity concentrations, which may be altered by several diseases, including Parkinson disease and attention deficit hyperactivity disorder (ADHD). Exemplary SPECT systems equipped with a USCB/slant collimating arrangement may also provide improved detection and activity quantification in tumors or other brain structures.

### BRIEF DESCRIPTION OF THE DRAWINGS

The foregoing and other objects of the present invention will be apparent upon consideration of the following detailed description, taken in conjunction with the accompanying drawings, in which:

FIG. 1a is an exemplary graph of transverse sensitivity profiles along a transaxial direction for several types of collimating arrangements, scaled by a sensitivity value for a conventional low-energy, high-resolution (“LEHR”) collimating arrangement at x=0 generated using an exemplary embodiment of the system and method according to the present invention;

FIG. 1b is an exemplary graph of axial sensitivity profiles for several types of collimating arrangements, scaled by a sensitivity value for an LEHR collimating arrangement at z=0 generated using an exemplary embodiment of the system and method according to the present invention;

FIG. 1c is an exemplary graph of sensitivity profiles along a 45° diagonal line in a coronal section for several types of collimating arrangements, scaled by a sensitivity value for an LEHR collimating arrangement at s=0 generated using an exemplary embodiment of the system and method according to the present invention;

FIG. 2a is an exemplary graph of predicted noise profiles (e.g., a standard deviation) along a transaxial direction for the collimating arrangements shown in FIG. 1a, scaled by the noise value for an LEHR collimating arrangement at x=0 generated using an exemplary embodiment of the system and method according to the present invention;

FIG. 2b is an exemplary graph of predicted noise profiles (standard deviation) in the axial direction for the collimating arrangements shown in FIG. 1b, scaled by the noise value for an LEHR collimating arrangement at z=0 generated using an exemplary embodiment of the system and method according to the present invention;

FIG. 2c is an exemplary graph of predicted noise profiles (standard deviation) along a 45° diagonal line in a coronal section for the collimating arrangements shown in FIG. 1c, scaled by the noise value for an LEHR collimating arrangement at s=0 generated using an exemplary embodiment of the system and method according to the present invention;

FIG. 3a is a transverse view of a system in accordance with an exemplary embodiment of the present invention which includes a cone-beam collimating arrangement having a focal length of about 20 cm and a fan-beam collimating arrangement having a focal length of about 40 cm;

FIG. 3b is an axial view of the exemplary system shown in FIG. 3a;

FIG. 4 is an exemplary graph of a Cramer-Rao bound-based signal-to-noise ratio (SNR_{CRB}) for estimation of an activity of spheres within an elliptical phantom embedded at 7 locations shown in an inset diagram generated using an exemplary embodiment of the system and method according to the present invention;

FIG. 5a is an exemplary illustration of a central transaxial slice through an axial center of an ellipsoidal phantom and reconstructed images thereof obtained using LEHR+LEHR, c40-f40, and c20-f40 collimating arrangement pairs generated using an exemplary embodiment of the system and process according to the present invention;

FIG. 5b is an exemplary illustration of a transaxial slice through striata of a Zubal phantom with an activity distribution characteristic of I-123-Altropane and reconstructed images thereof obtained using LEHR-LEHR, c40-f40, and c20-f40 collimating arrangement pairs generated using an exemplary embodiment of the system and process according to the present invention;

FIG. 6 is an exemplary illustration of several transaxial slices of a Zubal phantom with activity distribution characteristic of Tc-99m-HMPAO, and reconstructed images of these slices obtained using LEHR-LEHR, c40-f40, and c20-f40 collimating arrangement pairs;

FIG. 7 is an exemplary graph of SNR_{CRB} vs. distance between a center of a phantom and a center of an exemplary system containing spheres at the seven locations within an ellipsoidal phantom shown in FIG. 4 obtained using a c20-f40 collimating arrangement pair;

FIG. 8 is an exemplary illustration of a transaxial view of an exemplary hybrid collimating arrangement according to an exemplary embodiment of the present invention;

FIG. 9 is an exemplary graph of transverse sensitivity profiles for several collimating arrangements relative to the sensitivity of an LEHR collimating arrangement at x=0 generated using an exemplary embodiment of the system and method according to the present invention;

FIG. 10a is an exemplary illustration of a hole pattern at the center of a collimating arrangement having a focal length of about 50 cm generated using an exemplary embodiment of the system and method according to the present invention;

FIG. 10b is an exemplary illustration of a hole pattern approximately 15 cm from the center of the collimating arrangement of FIG. 10a;

FIG. 10c is an exemplary illustration of a hole pattern at the center of a collimating arrangement according to an exemplary embodiment of the present invention having a focal length of about 20 cm;

FIG. 10d is an exemplary illustration of a hole pattern approximately 15 cm from the center of the collimating arrangement of FIG. 10c;

FIG. 11a is an exemplary diagram of a conventional collimating arrangement (CC) which may be formed using casting techniques;

FIG. 11b is an exemplary diagram of a collimating arrangement having a uniform hole distribution (FC) in accordance with a certain exemplary embodiment of the present invention;

FIG. 11c is an exemplary diagram of a collimating arrangement having a uniform hole distribution and tapered holes (TC) in accordance with another exemplary embodiment of the present invention;

FIG. 12 is an exemplary schematic diagram of a geometrical configuration and associated parameters that may be used to calculate resolution of the collimating arrangement;

FIG. 13 is an exemplary graph of sensitivity values for three collimating arrangement configurations shown in FIGS. 11a-11c for a point source located at y₀=10 cm over a range of focal lengths f;

FIG. 14a is an exemplary graph of sensitivity values for the three collimating arrangement configurations shown in FIGS. 11a-11c, each having a focal length of about 20 cm, for various point source positions in a direction perpendicular to the surface of collimating arrangement;

FIG. 14b is an exemplary graph of sensitivity values for the three collimating arrangement configurations shown in FIGS. 11a-11c, each having a focal length of 20 cm, for point source positions that vary in a transverse direction from the center of focal line (at y₀=10 cm);

FIG. 15 is an exemplary diagram of a system in accordance with an exemplary embodiment of the present invention which includes a cone-beam collimating arrangement associated with a first detector, a fan-beam collimating arrangement associated with a second detector, and a processing arrangement configured to generate image data based on signals received from the detectors; and

FIG. 16 is an exemplary flow diagram of an exemplary method in accordance with certain embodiments of the present invention.

Throughout the figures, the same reference numerals and characters, unless otherwise stated, are used to denote like features, elements, components or portions of the illustrated embodiments. Moreover, while the present invention will now be described in detail with reference to the figures, it is done so in connection with the illustrative embodiments.

### DETAILED DESCRIPTION OF EXEMPLARY EMBODIMENTS OF THE INVENTION

A geometric sensitivity can represent a ratio of the number of photons detected to the number emitted from a source. When the attenuation is negligible, the sensitivity of a parallel-hole collimating arrangement may be independent of a source position, while that of a converging collimating arrangement can be position-dependent. A sensitivity of converging collimating arrangements may be greater than that of parallel collimating arrangements, and cone-beam collimating arrangements can provide greater sensitivity than fan-beam collimating arrangements having the same focal length. The sensitivity of both fan- and cone-beam collimating arrangements can depend on their focal lengths.

The sensitivity of several collimating arrangements can be determined based on analytic collimating arrangement aperture functions such as those described in S. Genna et al., “Annular single-crystal emission tomography systems” in The fundamentals of PET and SPECT (Wernick M N and Asrsvold J N), Academic Press, 2004 (in press); C. E. Metz et al., “The geometric transfer function for scintillation camera collimating arrangements with straight parallel holes,” Phys. Med. Biol. 25, 1059-1070 (1979); and B. M. W. Tsui and G. T. Gullberg, “The geometric transfer function for cone and fan beam collimating arrangements,” Phys. Med. Biol. 35, 81-93 (1990).

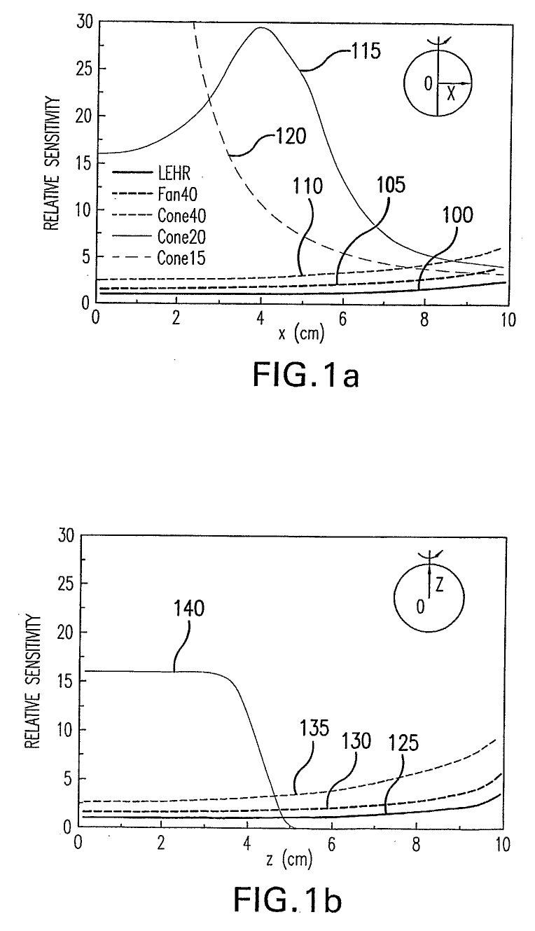

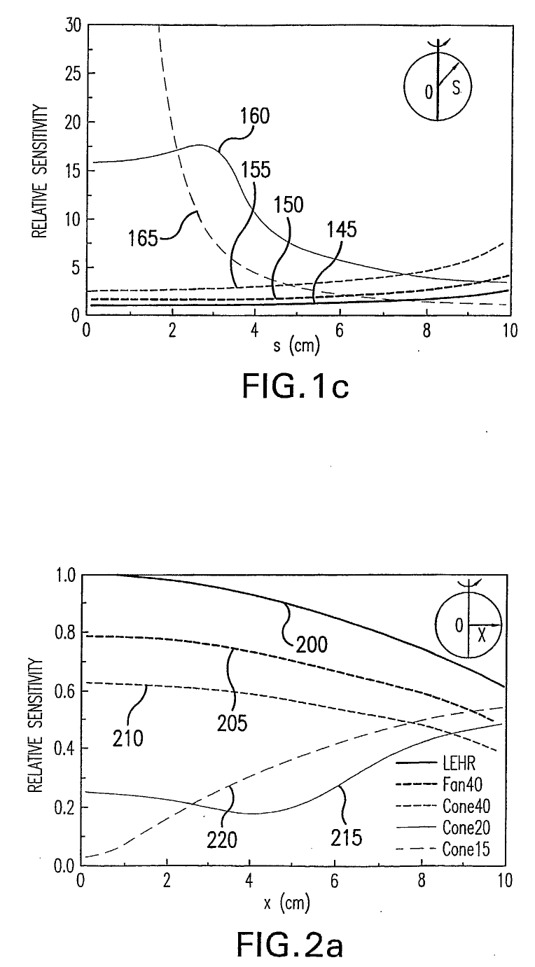

A relative sensitivity of several different collimating arrangements, summed over all projection angles, is shown as a function of point-source position in FIGS. 1a-1c. The sensitivity values shown in FIGS. 1a-1c can represent values that may be scaled by that of a conventional parallel low-energy, high-resolution (“LEHR”) collimating arrangement at x=0, z=0, and s=0, respectively, where x is a radial distance of a point source from a center of the imaging volume (“COV”) of a SPECT scanner. The COV can represent an intersection of the center of rotation (“COR”) and a central ray of the collimating arrangement. Sensitivity values were calculated by summing individual values over 120 projection angles covering 360 degrees. For these exemplary calculation, the radius-of-rotation (“ROR”) can be assumed to be 15 cm, measured from the COR to the nearest point on the collimating arrangement surface. The effects of attenuation may also included in these calculations. For example, a spherical attenuator can be centered on the COV, having a radius of 10 cm and a soft-tissue attenuation coefficient of about 0.15 cm⁻¹.

FIG. 1a is an exemplary graph of transverse sensitivity profiles along a transaxial direction generated with certain sections using the exemplary embodiments of the system and method of the present invention, and other profiles obtained using conventional collimating arrangements. The three lower curves represent the sensitivity of three conventional collimating arrangements: a parallel LEHR collimating arrangement 100, a fan-beam collimating arrangement with a 40-cm focal length 105, and a cone-beam collimating arrangement with a 40-cm focal length 110. The upper two curves 115, 120 correspond to cone-beam collimating arrangements according to exemplary embodiments of the present invention having shorter focal lengths of about 20 cm and 15 cm, respectively. The sensitivity values can be scaled by the sensitivity of an LEHR collimating arrangement at x=0. The relative sensitivity of the cone-beam collimating arrangement having a 15-cm focal length is 950 at the center (x=0), which is not shown in FIG. 1a.

FIG. 1b is an exemplary graph of axial sensitivity profiles for several collimating arrangements: a parallel LEHR collimating arrangement 125, a fan-beam collimating arrangement with approximately a 40-cm focal length 130, a cone-beam collimating arrangement with approximately a 40-cm focal length 135, and a cone-beam collimating arrangement having a focal length of approximately 20 cm 140. The sensitivity values in FIG. 1b are scaled by the sensitivity of an LEHR collimating arrangement at z=0. The axial sensitivity profile of the cone-beam collimating arrangement having approximately a 15-cm focal length is not shown because activity along the axis of rotation is not seen by this collimating arrangement, except at z=0.

FIG. 1c is an exemplary graph of sensitivity profiles along a 45° diagonal line in a coronal section for several collimating arrangements: a parallel LEHR collimating arrangement 145, a fan-beam collimating arrangement with approximately a 40-cm focal length 150, a cone-beam collimating arrangement with approximately a 40-cm focal length 155, a cone-beam collimating arrangement having a focal length of approximately 20 cm 160, and a cone-beam collimating arrangement having a focal length of approximately 15 cm 165. The sensitivity values in FIG. 1c are scaled by the sensitivity of an LEHR collimating arrangement at s=0.

The average attenuation length over all projection angles can be larger near the center (transaxially) than in the periphery away from a central axis. Thus, sensitivity of a parallel-hole collimating arrangement may be lower at the center (e.g., along the axis) than at or near the periphery. Collimation configurations which can provide higher sensitivity near the center may provide an improved imaging task performance throughout most of the imaging volume as compared with configurations characterized by constant sensitivity profiles. This exemplary effect is described, e.g., in the Kijewski publication. A centrally peaked sensitivity can compensate for larger attenuation paths from the center, which can provide an exemplary improved imaging performance over most of the brain or a specific portion thereof, and larger gains can be generated near the center. Because the converging collimating arrangements (e.g., cone-beam collimating arrangements) can provide increased sensitivity near the center, as shown in FIGS. 1a-1c, they can provide an imaging performance that can be better than the performance obtained using conventional collimating arrangements.

Short focal-length cone-beam collimating arrangements may provide high sensitivity gains near the center of the brain. Different values of the focal length may be evaluated, ranging from about 15 cm to 40 cm. Exemplary sensitivity profiles along radii in a reconstructed image are shown in FIGS. 1a-1c. A collimating arrangement which focuses at about 20 cm and which is about 5 cm beyond the COR, can provide a gain 115 that may be more than about 10 times greater than that of a parallel-hole collimating arrangement 100 out to a radial distance of about 6 cm in the transaxial direction, as shown in FIG. 1a. This exemplary collimating arrangement can also provide a gain 140 that may be more than about 10 times greater than that of a parallel-hole collimating arrangement 125 out to an axial distance of about 4 cm, as shown in FIG. 1b.

A collimating arrangement that focuses exactly to the center of the brain (e.g., having a focal length about f=ROR=15 cm), may provide a gain that can be significantly greater near the center. For example, the gain 120 may be up to about 950 times larger at the focal point, and may decrease dramatically with radial distance as shown in FIG. 1a. Furthermore, the points along the center of the rotation axis that do not lie in the plane of the central ray may not be seen by this collimating arrangement, as shown in FIG. 1b. A somewhat longer focal length (e.g., about f=20 cm) can provide an enhanced sensitivity 115, 140, 160 over a larger region of the brain, as shown in FIGS. 1a and 1b.

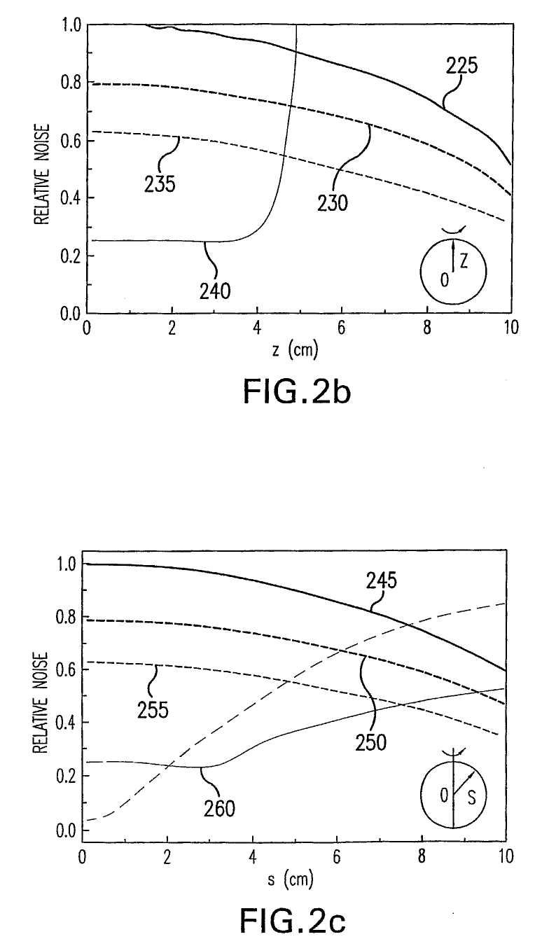

Count sensitivity can be an important characteristic of the SPECT imaging camera because systems having a high sensitivity may produce images which are less noisy than those obtained from low-sensitivity systems. Therefore, it may be useful to consider predicted relative image noise for the collimating arrangement shown in FIGS. 1a-1c. The relative noise, shown in FIGS. 2a-2c, may be estimated as being inversely proportional to the square root of the total number of photons detected over about 120 projection angles from a given source radial position. In FIGS. 2a-2c, as in FIGS. 1a-1c, the curves may be normalized by a corresponding value for a parallel-hole LEHR collimating arrangement associated with a source positioned at the COV.

For example, FIG. 2a shows an exemplary graph of predicted noise profiles along a transaxial direction for the collimating arrangements shown in FIG. 1a: a parallel LEHR collimating arrangement 200, a fan-beam collimating arrangement with about a 40-cm focal length 205, a cone-beam collimating arrangement with about a 40-cm focal length 210, a cone-beam collimating arrangement having a focal length of about 20 cm 215, and a cone-beam collimating arrangement having a focal length of about 15 cm 220. The noise values are scaled by the noise predicted for an LEHR collimating arrangement at x=0.

FIG. 2b shows an exemplary graph of predicted axial noise profiles for the collimating arrangements shown in FIG. 1b: a parallel LEHR collimating arrangement 225, a fan-beam collimating arrangement with about a 40-cm focal length 230, a cone-beam collimating arrangement with about a 40-cm focal length 235, and a cone-beam collimating arrangement having a focal length of about 20 cm 240. The noise values are scaled by the noise predicted for an LEHR collimating arrangement at z=0.

FIG. 2c shows an exemplary graph of predicted noise profiles along a 45° diagonal line in a coronal section for the collimating arrangements shown in FIG. 1c: a parallel LEHR collimating arrangement 245, a fan-beam collimating arrangement with about a 40-cm focal length 250, a cone-beam collimating arrangement with about a 40-cm focal length 255, a cone-beam collimating arrangement having a focal length of 20 cm 260, and a cone-beam collimating arrangement having a focal length of 15 cm 265. The noise values are scaled by the noise predicted for an LEHR collimating arrangement at s=0.

Ultra-short focal-length cone-beam collimating arrangements (e.g., those having a focal length of about f=15 cm or 20 cm) may produce images with lower noise 220, 215 over most of the field of view (e.g., |x|<8 cm transaxially) as compared to conventional collimating arrangements 200, 205, as shown in FIG. 2a. A cone-beam collimating arrangement having a focal length of about 20 cm may also generate with lower noise 240 for |z|<4 cm in an axial direction as compared to conventional collimating arrangements 225, 230 as shown in FIG. 2b.

A cone-beam collimating arrangement having about a 15-cm focal length may produce less noise 265 than a cone-beam collimating arrangement having a focal length of about 20 cm 260 for a small region within about ˜2 cm of the COV, as shown in FIG. 2c. However, the 20-cm cone-beam collimating arrangement can provide noise levels 260 that may be more uniform and lower than those provided by the 15-cm collimating arrangement 265 over a much larger volume of the brain or a specific portion thereof. In the small region within ˜2 cm of the COV, where the 15-cm collimating arrangement may yield lower noise 265 than the 20-cm collimating arrangement 260, the 20-cm collimating arrangement may provide a level of image noise 260 that is approximately 4 times lower than that of a conventional low-energy, high-resolution (LEHR) collimating arrangement 245, and about 3 times lower than that of a fan-beam collimating arrangement 250. For example, a 20-cm focal length collimating arrangement may be selected for further evaluation because it can provide a reasonable compromise between the sensitivity gain and the volume to be imaged in which an increased sensitivity can be achieved.

Exemplary projections that can be used to create artifact-free SPECT reconstructions may need to satisfy a data sufficiency condition as described, e.g., in H. K. Tuy, “An inversion-formula for cone-beam reconstruction,” SIAM J. Appl. Math. 43, 546-552 (1983). Brain SPECT camera orbits obtained using conventional parallel- and fan-beam collimation may provide sufficient data. However, with a single circular orbit with cone-beam collimation, it may be difficult to provide artifact-free SPECT images because the cone-beam projections data may not satisfy a sufficiency condition as described in B. D. Smith, “Image Reconstruction from cone-beam projections: necessary and sufficient conditions and reconstruction methods,” IEEE Trans. Med. Imag. MI-4:14-25 (1985). Under these conditions, the reconstructed homographic images may appear increasingly distorted with increasing axial distances from the central section. With conventional, long focal-length cone-beam collimation, these distortions may be acceptable, e.g., when homographic images are reconstructed iteratively. When using ultra-short focal-length collimation as described herein, the effects of a data truncation may be too severe. Such data may be truncated in both the axial and transaxial directions, which can further exacerbate geometric distortions and artifacts.

Certain techniques for reducing or eliminating axial distortions in conventional cone-beam collimation can prefer modification of the SPECT camera orbit, and can be accomplished by moving a patient table during the SPECT acquisition as described, for example, in G. T. Gullberg et al., “Review of convergent beam tomography in single photon emission computed tomography,” Phys. Med. Biol., 37, 507-534 (1992). This exemplary technique may be impractical for brain SPECT procedures because, e.g., the cameras may not clear a patient's shoulders if the table is moved further into a gantry to complete the projection data.

Another approach, as described in R. J. Jaszczak et al., “3-Dimensional SPECT reconstruction of combined cone beam and parallel beam data,” Phys. Med. Biol. 37, 535-548 (1992), can include simultaneous collection of parallel- and cone-beam projection data on two heads of a dual-detector SPECT apparatus in order to suppress artifacts related to data insufficiency. In this exemplary technique, a parallel-hole collimating arrangement can provide sampling of regions of Radon space that may not be sampled by a simple planar orbit using only a cone-beam collimation. A fan-beam collimating arrangement can provide sufficient data for homographic reconstruction of brain SPECT images with a higher count sensitivity than that obtained using a parallel-hole collimating arrangement. Thus, a combination of a 20-cm cone-beam collimating arrangement on one camera head and a 40-cm fan-beam collimating arrangement on a second camera head may be used to obtain improved images. A 40-cm focal length can be long enough to ensure that the fan-beam projections are not truncated in a transaxial direction. In this exemplary system according to one exemplary embodiment of the present invention, the fan-beam collimating arrangement can provide complete projection data, whereas the ultra-short cone-beam collimating arrangement can provide greatly increased sensitivity over most of the imaging FOV.

When using a dual-head SPECT camera instrument, it may be difficult to position the brain axially at the center of the FOV while still maintaining a close detector orbit (small ROR) in order to achieve optimal collimating arrangement resolution. This is because the patient's shoulders preferably should not be contacted by the cameras during their rotation. Several approaches have been described for circumventing this problem. For example, if the cone-beam collimating arrangement is focused on or near the axial center of the cameras, the shoulders can be cleared by tilting the camera heads as described, e.g., in Jaszczak et al. This exemplary technique may permit detector orbits that are close to regions of the brain of primary interest while yielding degraded resolution near the cerebellum. Data sufficiency of a fan-beam collimating arrangement may be maintained in this technique, for example, by using a tilted fan-beam collimating arrangement with an axial tilt angle equal and opposite to the camera tilt angle.

To avoid degrading resolution, a half-cone-beam such as that described in the Li publication, or a shifted-center cone-beam collimation as described in the Kamphuis publication and in the Lalush publication, may be used. In exemplary embodiments of the present invention, a cone-beam collimating arrangement can be provided having a focal point that is shifted down towards the body (caudally), which may allow the camera to clear the shoulders while maintaining about a 15-cm camera ROR.

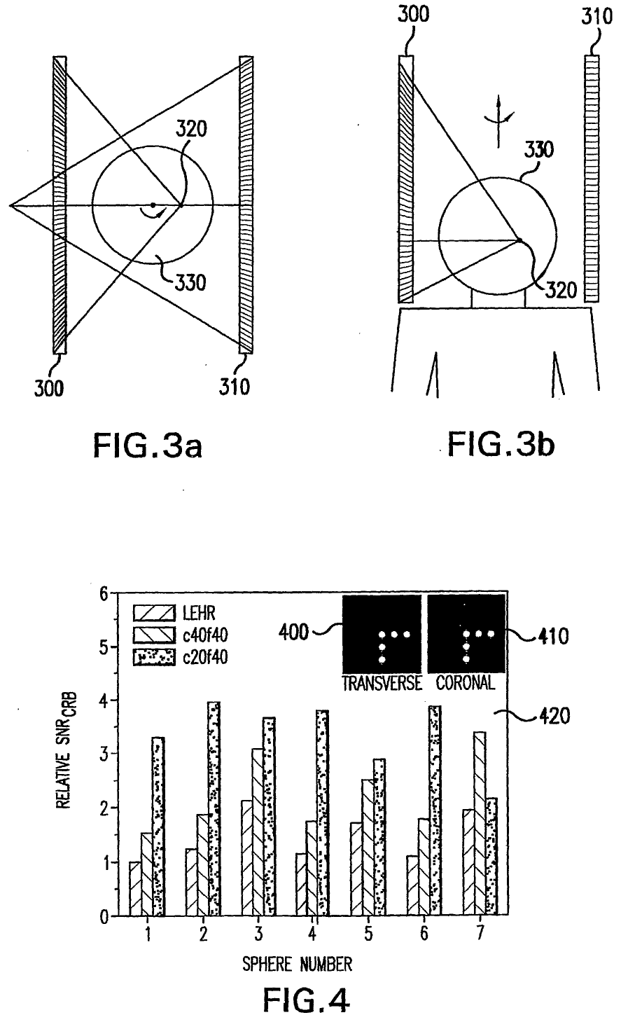

Transaxial and axial views of such an exemplary system are shown in FIGS. 3a and 3b, respectively. For example, the focal point 320 of the cone beam collimating arrangement 300 which is located, e.g., 20 cm from the collimating arrangement, is located within a brain 330 being imaged. The camera dimensions in this scale drawing are approximately 52 cm transaxially by about 38 cm axially. The brain size may be represented as an ellipsoid with a major-axis length of about 21 cm and a minor-axis length of about 17.6 cm. The fan-beam collimating arrangement 310 can collect complete data for artifact-free homographic reconstruction. The focal point 320 of the cone-beam collimating arrangement 300 can be shifted about 9 cm inferiorly from the center of the camera in the axial direction, as shown in FIG. 3b. The focal point of a half-cone-beam collimating arrangement may prefer a shift by an even greater axial distance, which can lead to greater hole angulation at a superior end of the collimating arrangement and an increased difficulty in fabricating such collimating arrangement.

Several pairs of collimating arrangements may be evaluated by simulation and analysis, and compared on the basis of performance in an activity estimation task. These quantitative assessments can also be compared to a rank-ordering of image quality. Noise-free images of phantoms may also be inspected for evidence of truncation artifacts or aliasing due to an insufficient sampling.

Collimating arrangement pairs that were evaluated included either an LEHR collimating arrangement or a fan-beam collimating arrangement on a first detector head in order to yield a complete data set. These collimating arrangements were paired with an LEHR collimating arrangement, a fan-beam collimating arrangement, or a cone-beam collimating arrangement. The converging collimating arrangements evaluated may be denoted by the following abbreviations: “f40” refers to a fan-beam collimating arrangement with a focal length of 40 cm, and “c20” and “c40” refers to cone-beam collimating arrangements having focal lengths of 40 cm and 20 cm, respectively. For all collimating arrangement combinations evaluated, the distance from the COR to the surface of the collimating arrangements was fixed at 15 cm to simulate a circular orbit. The collimating arrangements were all assumed to be 2.4 cm thick, with 1.1-mm-diameter holes and a septal thickness of 0.16 mm on the surface of the collimating arrangement closest to the patient. These values may be typical for a conventional LEHR parallel-hole collimating arrangement. The hole size and septal thickness of converging collimating arrangements may increase off-axis, e.g., with increasing distance from a collimating arrangement hole that is perpendicular to a detector, because of obliquity effects.

All collimating arrangements evaluated may have identical or substantially similar central-ray geometric hole parameters. This assumption can be made for two reasons. First, collimating arrangement manufacturers may have a limited number of sets of pins to be used for casting lead collimating arrangements, so it can be more straightforward and less costly to fabricate collimating arrangements with one of these standard hole sizes, rather than re-machining new pins. Second, the assumption of equal hole size can lead to increasingly conservative estimates of imaging task performance with increasing degrees of collimating arrangement convergence. Converging holes may provide improved collimating arrangement resolution, as well as improved intrinsic camera spatial resolution, because of image magnification. Therefore, a comparison of imaging performance using converging collimation to that obtained using LEHR parallel collimation for equal spatial resolution at a particular or arbitrary point within the object can prefer an increase in the hole size of the converging collimation, a decrease of the septal thickness, or a decrease of the collimating arrangement thickness. In addition to altering the penetration characteristics of the collimating arrangements, any of these variations can lead to even higher count sensitivity than is assumed for the performance assessments described herein. Therefore, the gains from converging collimation described herein using the assumption of identical or substantially similar central-ray hole parameters may underestimate the actual performance gains.

Two phantoms were used to evaluate various collimating arrangement pairs. The first phantom studied was an ellipsoid having semi-axes of 8.8 cm (x-direction) and 10.5 cm (y-direction) transaxially, and 8.8 cm axially (z-direction), containing a uniform-background activity concentration, together with one or more spheres, each having a diameter of 0.9 cm and an activity concentration that is four times higher than that of the background. These spheres were located in several different positions along the axes. The sphere locations in the central transaxial slice 400 and the central sagittal slice 410 are shown in FIG. 4.

The second phantom reviewed was a Zubal brain phantom as described, e.g., in I. G. Zubal et al., “Computerized 3-dimensional segmented human anatomy,” Med. Phys. 21, 299-302 (1994). This phantom was used to simulate two activity distributions. The first of these distributions may be characteristic of I-123-Altopane, a dopamine transporter tracer that concentrates in the striata, centrally located structures. The second distribution simulated may be similar to that of Tc-99m-HMPAO, a perflision tracer. In both activity distributions reviewed, normal distributions were simulated. The first distribution allowed an evaluation of an image quality near the center of the brain, while the second distribution allowed assessment of image quality throughout the brain.

Imaging data were simulated by ray-tracing through voxelized source distributions, and attenuation of photons was modeled using narrow-beam attenuation coefficients at 140 keV. Collimated ray-sums were blurred and scaled using conventional expressions for collimating arrangement resolution and sensitivity as described, e.g., in S. C. Moore et al., “Collimating arrangement design for single photon-emission tomography,” Eur. J. Nucl. Med. 19, 138-150 (1992). An intrinsic resolution of the detector was assumed to be about 3.2 mm FWHM. A resolution degradation with increasing source-collimating arrangement distance was modeled for all collimating arrangements. The sensitivity of the LEHR collimating arrangement did not vary with spatial location, while that of the converging collimating arrangements depended on both the collimating arrangement angle of incidence and the distance from the source position to the focal point.

Exemplary simulated projection data may be constructed from both the ellipsoidal phantom with spheres located as shown in FIG. 4, and the Zubal brain phantom. Poisson-distributed pseudorandom noise was added to each projection pixel, and the noisy data were reconstructed iteratively using an OS-EM algorithm as described in H. M. Hudson, R. S. Larkin, “Accelerated image-reconstiruction using ordered subsets of projection data,” IEEE Trans. Med. Imag. 13, 601-609 (1994). Exemplary parameters used for this reconstruction include 20 subsets, 6 projections per subset and 4 iterations, with attenuation modeled in the projector/back projector. The exemplary reconstruction technique was written so that it could be used with a variety of simulated data, including that obtained from both parallel-hole and converging collimating arrangements. Images were reconstructed onto a 128×128×128 matrix with 1.8×1.8×1.8 mm³ voxels.

The collimating arrangement pairs were evaluated based on performance of activity estimation. A signal-to-noise ratio, SNR_{CRB} was determined based on the Cramer Rao lower bound (“CRB”) on variance of the activity estimates, for estimation of activity concentration within a lesion. The CRB was determined for each sphere from the following model of the projection:

I(θ,x,z)=A₁sph(θ,x,z)+A₂f(θ,x,z) (1)

where sph(θ,x,z) can represent a projection of the sphere at detector position (x,z) and projection angle θ, calculated by ray-tracing as described herein above, and f(θ,x,z) can represent a projection of the ellipsoidal background. The two unspecified parameters in this expression are the activity concentrations, A₁ and A₂, of the sphere and the background respectively. The size and location of each sphere is known. The CRB on the variance of sphere activity concentration estimates can be represented by [J⁻¹]₁₁, where J, Fisher's information matrix, can be expressed as

$${J}_{ij} = {\sum }_{detector}^{ } {\sum }_{θ , x , z}^{ } ( \frac{\partial I ( θ , x , z )}{\partial {A}_{i}} ) ( \frac{\partial I ( θ , x , z )}{\partial {A}_{j}} ) \cdot \frac{1}{I ( θ , x , z )} , \qquad ( 2 )$$

and is described, e.g., in H. Van Trees, Detection, Estimation and Modulation Theory. New York, Wiley (1968). The corresponding signal-to-noise ratio SNR_{CRB} can be expressed as

$$S N {R}_{C R B} = \frac{{A}_{1}}{\sqrt{C R B ( {A}_{1} )}} = \frac{{A}_{1}}{\sqrt{{[ {J}^{- 1} ]}_{11}}} . \qquad ( 3 )$$

The SNR_{CRB} for a sphere was calculated at seven locations for several collimating arrangement pairs.

In the evaluations of collimating arrangement pairs described herein above, the center of the brain was assumed to coincide with the center of the system, which may be defined as an intersection between the axis of rotation and the central ray of the converging collimating arrangements. Because such precise positioning may be difficult to achieve in a clinical setting, the effects of mismatches on sensitivity for two collimating arrangement pairs, c40-f40 and c20-f40, were evaluated. This evaluation was performed by shifting the center of the brain in three orthogonal directions by up to 8 pixels (approximately 14.4 mm) relative to the center of the system in order to simulate inaccurate patient positioning, and calculating the resulting variations in SNR_{CRB} values.

For the seven sphere locations evaluated, SNR_{CRB} was greater for the converging collimating arrangement pairs, c40-f40 and c20-f40 than for the LEHR pair, as shown in the graph 420 of FIG. 4. For example, for all but the most extreme axial location corresponding to sphere number 7, for which the photons from the sphere may not be detected by the collimating arrangement c20 having a 20 cm focal length, SNR_{CRB} was higher for the c20-f40 collimating arrangement pair than for the c40-f40 collimating arrangement pair. The greatest gains in SNR_{CRB}, were achieved at locations within about 6 cm from the center (e.g., spheres 1, 2, 4, and 6 of FIG. 4). This exemplary result may be consistent with the combined sensitivity profiles provided in FIGS. 1a-1c for the cone collimating arrangement having a 20 cm focal length and the fan collimating arrangement having a 40 cm focal length. At these locations, SNR_{CRB} values achieved using a c20-f40 collimating arrangement pair exceeds that achieved with an LEHR collimating arrangement by a factor of about 3, and exceeds that achieved using a c40-f40 collimating arrangement pair by a factor of about 2. Therefore, a short-focal-length cone beam/fan beam collimating arrangement set can provide a better task performance over most of the brain, and somewhat better performance in the periphery, than a conventional LEHR collimating arrangement pair.

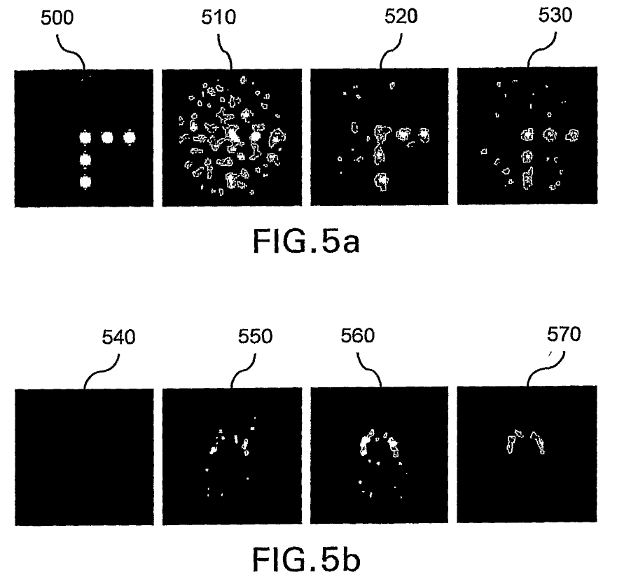

Exemplary transverse images of a central slice of the sphere phantom 500 are shown in FIG. 5a for several collimating arrangement pairs. FIG. 5a shows an exemplary image obtained using an LEHR collimating arrangement pair 510, an image obtained using a c40-f40 collimating arrangement pair 520, and an image obtained using a c20-f40 collimating arrangement pair 530. The number of detected counts was 132,000 for the LEHR collimating arrangement pair 510. This can be a typical count level for I-123-Altropane studies. The number of detected counts was 290,000 for the c40-f40 collimating arrangement pair 520, and 360,000 for the c20-f40 collimating arrangement pair 530. The results shown in FIG. 5a indicate that the converging collimation can yield an improved sphere visibility as compared to parallel-hole collimation. The c20-f40 collimating arrangement combination 530 yielded the lowest noise levels and, consequently, the most clearly visible spheres within a distance of about 6 cm from the center in a transaxial direction.

Transverse slices of the Zubal phantom with an I-123-Altropane activity distribution 540 are shown in FIG. 5b for several collimating arrangement pairs. FIG. 5b shows an exemplary image of the Zubal phantom 540 obtained using an LEHR collimating arrangement pair 550, an image obtained using a c40-f40 collimating arrangement pair 560, and an image obtained using a c20-f40 collimating arrangement pair 570. The improved image quality obtained with the c20-f40 collimating arrangement combination 570 for a striatal imaging can be seen in FIG. 5b.

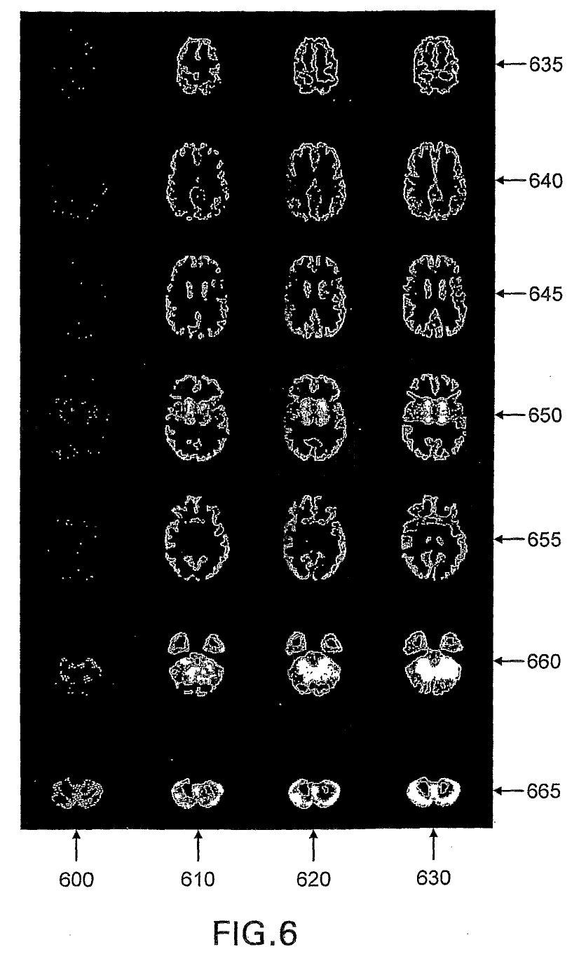

Seven transverse slices 635-665 of the exemplary Zubal phantom 600 with a Tc-99m-HMPAO activity distribution are shown in FIG. 6. The set of images 610 were obtained using an LEHR collimating arrangement pair, the images 620 were obtained using a c40-f40 collimating arrangement pair, and the images 630 were obtained using a c20-f40 collimating arrangement pair. The images in the top and bottom rows 635, 665 are about 5 cm above and below the focal point, respectively. The images in the second from the top and second from the bottom rows 640, 660 are about 3 cm above and below the focal point, respectively. For all regions of all slices, the focusing collimating arrangements 620, 630 yielded lower-noise images than the LEHR collimating arrangement pair 610. For the central regions of the middle three slices 645-655, the c20-f40 collimating arrangement pair 630 yielded the lowest noise images. These images illustrate an improvement in imaging the center of a brain that can be obtained using a very short focusing collimating arrangement.

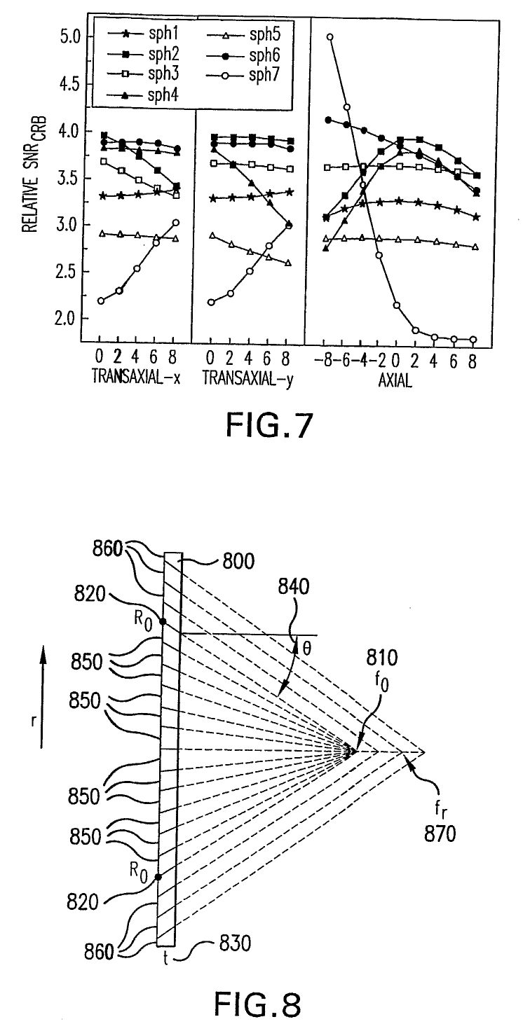

The SNR_{CRB} values for the c40-f40 and LEHR collimating arrangement pairs were observed to be minimally affected by shifting the center of the brain up to 14.4 mm from the intersection of the center of rotation and the central ray of the collimating arrangements. FIG. 7 shows an exemplary graph of relative SNR_{CRB} values for a phantom containing seven spheres (e.g., sph1-sph7) in the locations 400, 410 shown in FIG. 4. The SNR_{CRB} values in FIG. 7 were obtained using a c20-f40 pair collimating arrangement as a function of spatial shifts in three orthogonal directions (e.g., transaxial x and y directions, and axial direction). The SNR_{CRB} values for spheres near the focal point were observed to be more sensitive to such a shift than for those farther from the focal point. For example, the SNR_{CRB} values observed for sphere 1, located at the center of the phantom, varied little (by less than about 10%) with respect to a shift in any direction as compared to the values measured for other lesions (e.g., spheres 2-7). The peripherally-located spheres were observed to be most sensitive to shifts in the phantom location relative to the collimating arrangements. For example, the SNR_{CRB} value for sphere 7 was observed to vary significantly with axial shifting. However, all sensitivity values obtained using a c20-f40 collimating arrangement pair were observed to be greater than the corresponding sensitivity values obtained using a c40-f40 collimating arrangement pair or an LEHR collimating arrangement pair over the range of shifts shown in FIG. 7. Thus a c20-f40 collimating arrangement pair may provide improved imaging performance as compared to conventional collimating arrangement pairs even with respect to the mismatches between the center of the brain and the center of the system.

Very short focal-length cone-beam collimating arrangements, such as the one described herein for high-sensitivity brain imaging, may be difficult to manufacture. Conventional manufacturing techniques such as pouring molten lead around pins may not be appropriate because of the large hole angles (up to 59 degrees for the 20-cm focal-length collimating arrangement) required near the collimating arrangement edges. Very short focal-length cone-beam collimating arrangements may be fabricated using other approaches such as etching, as described in R. H. Moore et al., “A variable angle slant-hole collimating arrangement,” J. Nucl. Med. 24, 61-65 (1982), or stamping as described, e.g., in S. Genna, J. Ouyang, and W. Xia, “Annular single-crystal emission tomography systems” in The fundamentals of PET and SPECT (Wernick M N and Asrsvold J N), Academic Press, 2004, whereby collimating arrangement layers can be shaped and subsequently stacked to form a complete collimating arrangement.

Exemplary embodiments of the present invention can be implemented using a ultra-short cone-beam (USCB) collimating arrangement. Because the angle between many of the holes in a USCB collimating arrangement and the vector normal to the collimating arrangement surface may be very large, it may be difficult to use conventional manufacturing methods such as casting lead or stamping and stacking lead foils to produce such collimating arrangements.

In accordance with another exemplary embodiment of the present invention, it may be possible to utilize, e.g., a hybrid ultra-short cone-beam/slant-hole (USCB/S) collimating arrangement, which may provide an increased sensitivity and would not require hole angles larger than about 38 degrees. For example, such a hybrid collimating arrangement can be manufactured using a conventional casting technique and it may not require a dedicated SPECT system. Therefore, implementing the USCB/S collimating arrangement in existing dual-head SPECT systems or components may be cost efficient.

In addition to using the USCB collimating arrangements in a dual-head SPECT system as described herein above, USCB collimation can also be used with, e.g., a triple-head SPECT system. For example, two USCB/S collimating arrangements and a fan-beam collimating arrangement can be used in a triple-head SPECT system which may provide a larger gain in a sensitivity than can be achieved with a dual-head system.

FIG. 8 shows a transaxial view of a hybrid collimating arrangement 800 according to one exemplary embodiment of the present invention with f₀ being the focal length 810 of the focusing portion of the collimating arrangement, which extends to a radial distance R₀ 820, and “t” 830 being a collimating arrangement thickness. The hybrid collimating arrangement 800 can be a combination of USCB and slant-hole collimating arrangements. Holes 850 near the center of the collimating arrangement can be focused, e.g., to 20 cm (or, more generally, f₀ 810) in front of the surface out to R₀, where the hole angle θ 840 can be, e.g., approximately 38 degrees. The slanted holes 860 that are located further from the center of the collimating arrangement can be formed at this constant angle θ 840, as shown in FIG. 8. Therefore, the collimating arrangement 800 can be manufactured using conventional lead casting techniques. The sensitivity gain of the hybrid collimating arrangement 800 may not be as large as that of the USCB collimating arrangement, but it may have an extended field of view. For example, the slanted holes 860 may have an angle of 38 degrees with respect to the surface normal. Therefore, they can point to fᵣ 870, depending on the radial distance r from the center of the collimating arrangement plane. In certain exemplary embodiments of the present invention, the hole angle θ 840 selected for a USCB/S collimating arrangement may be greater or less than about 38 degrees. This angle can be selected based on, e.g., the relative importance of sensitivity and field of view when imaging a particular organ.

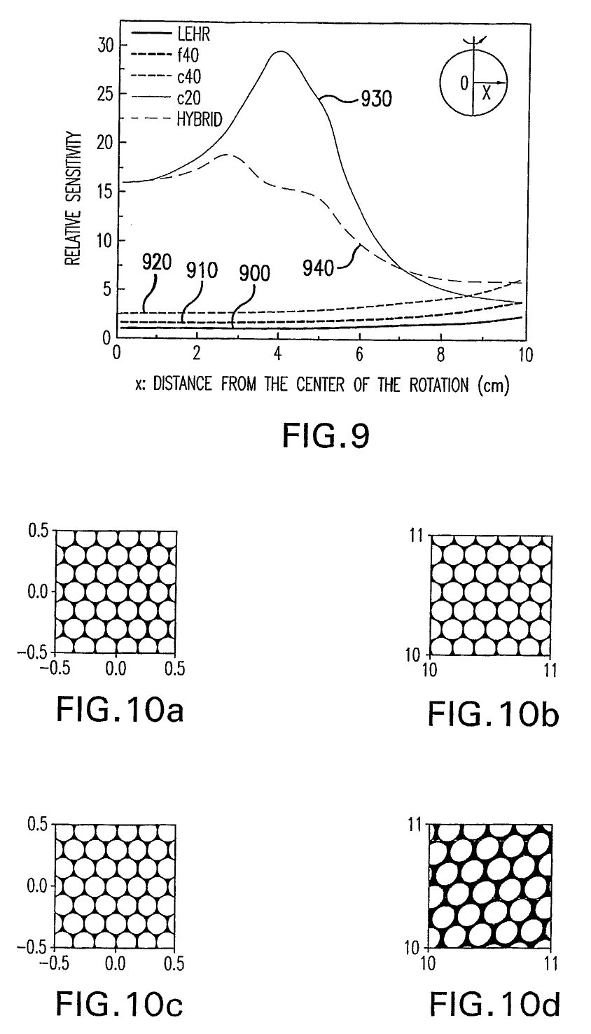

A centrally peaked sensitivity can compensate for the larger attenuation paths from the center, which may provide improved imaging performance over most of the brain and largest gains near the center. Characterizing the sensitivity of a collimating arrangement can be an important procedure for designing a collimating arrangement. FIG. 9 shows a graphic of a relative transverse sensitivity of several different collimating arrangements as a function of point-source radial location: a parallel LEHR collimating arrangement 900, a fan-beam collimating arrangement with about a 40-cm focal length 910, a cone-beam collimating arrangement with about a 40-cm focal length 920, a cone-beam collimating arrangement having a focal length of 20 cm 930, and a hybrid USCB/S collimating arrangement 940. The sensitivity values are scaled by the sensitivity of an LEHR collimating arrangement at x=0.

Because the average attenuation length over the projection angles is larger near the center (transaxially) than in the periphery, the sensitivity obtained with conventional collimating arrangements may be lower at the center than in the periphery. However, the USCB collimating arrangement can collect more photons originating near the center, which may tend to compensate for this attenuation. The exemplary hybrid USCB/S collimating arrangement described herein which may be used in exemplary embodiments of the present invention can yield a sensitivity gain that is lower than that of a USCB collimating arrangement having a focal length of about 20 cm. The sensitivity gain that may be achieved using the hybrid USCB/S collimating arrangement averaged over all regions can be about 10 times greater than the sensitivity gain of a conventional parallel-hole collimating arrangement, and can be about 14 times greater if the sensitivity gain values are averaged out to a radial distance of about 6 cm in the transaxial direction, where many interesting brain structures can be located. For example, the higher sensitivity of the exemplary USCB/S collimating arrangement described herein can allow more precise estimates of striatal activity concentrations, which may be altered in several diseases including Parkinson disease and attention deficit hyperactivity disorder (ADHD). The SPECT systems equipped with the hybrid USCB/S collimating arrangement may also provide improved detection and activity quantification associated with tumors or other brain structures.

In further exemplary embodiments of the present invention, the USCB collimating arrangements that have another exemplary design may be used to further increase sensitivity gain. For example, a USCB design can prefer an angle between a hole near the periphery and a vector normal to the collimating arrangement surface to be larger (e.g., up to about 50 degrees) than the maximum achievable by conventional manufacturing methods (e.g., approximately 38 degrees) such as casting lead or stamping and stacking lead foils. Moreover, a hexagonal close packing may not be achieved for peripheral holes in cone-beam collimating arrangements that are manufactured using casting techniques. As focal length decreases, gaps between peripheral holes can become larger. These exemplary designs and differences therebetween are illustrated in FIGS. 10a-d. FIGS. 10a and 10b show conventional hole patterns at the center and approximately 15 cm away from the center, respectively, of a collimating arrangement having a focal length of 50 cm. FIGS. 10c and 10d show hole patterns at the center and approximately 15 cm away from the center, respectively, of the USCB collimating arrangement having a focal length of 20 cm. Larger gaps are visible between peripheral holes in the USCB shown in FIG. 10d than in the conventional collimating arrangement shown in FIG. 10b. For these reasons, a casting technique may not be appropriate for USCB collimating arrangements. More flexible manufacturing approaches exist such as, for example, stacking photoetched tungsten/gold foils and growing septa using stereolithography (“SL”). Certain manufacturing techniques can preserve hexagonal close packing on the front surface of a collimating arrangement and may further allow for variations in collimating arrangement hole size with depth in the collimating arrangement. For example, tapered collimating arrangement holes may offer advantageous performance.

Three collimating arrangement hole patterns may be analyzed with respect to associated sensitivity gains in the brain SPECT imaging for focal lengths, f, ranging from about 20 to 50 cm. The hole size can be adjusted for each pattern so that average spatial resolution was constant for all sensitivity comparisons. For simplicity, circular holes may be modeled in the analysis described herein, rather than hexagonal holes. Details of the hole shape may not significantly affected the results of such an analysis as described, e.g., in C. E. Metz et al., Phys. Med. Biol., 25:1059-1070 (1980).

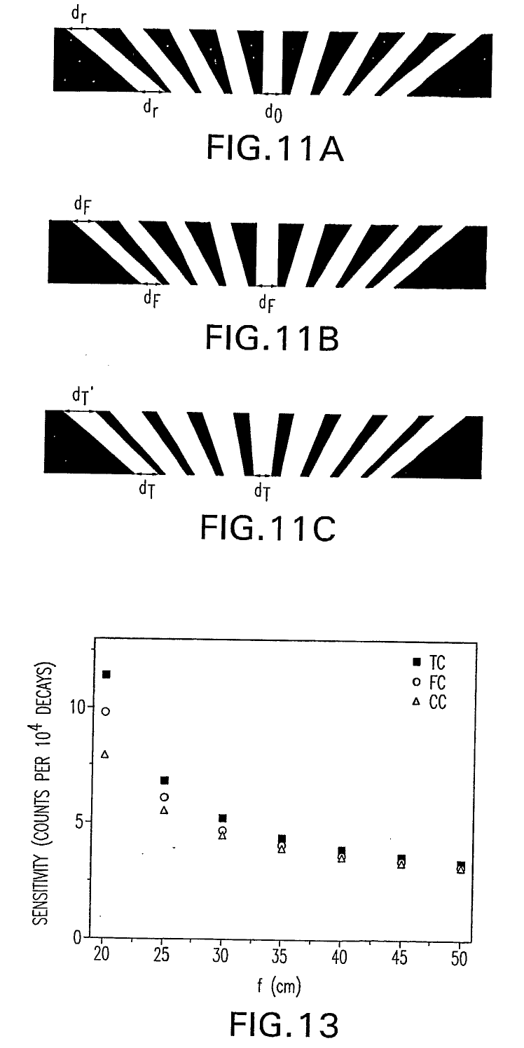

The exemplary collimating arrangement patterns described herein include: a collimating arrangement manufactured by a conventional casting technique (CC), having a hole size and septal thickness which increase with distance from the center, shown in FIG. 11a; a collimating arrangement having a hole size that is constant over the collimating arrangement surface and throughout the collimating arrangement (FC), shown in FIG. 11b; and a collimating arrangement having a hole size that is constant over the collimating arrangement surface and which increases with depth in the collimating arrangement (TC), shown in FIG. 11c.

The conventional CC collimating arrangement shown in FIG. 11a includes a hole size that is d₀ 1100 at the center; and which increases as the hole angle increases to a value dᵣ 1105 at a distance r from the center. This hole size is measured in a plane parallel to the front surface 1110 of the collimating arrangement. The hole size dᵣ 1105 can be determined by the expression dᵣ=d₀/cos θᵣ, where θᵣ is an angle between a hole axis and a normal to the collimating arrangement surface. The hole size measured in a plane perpendicular to the axis of a hole can be equal to d₀ for each hole in a CC collimating arrangement, because pins of constant diameter but configured at varying angles can be used to form these cast holes. If a minimal hole tapering preferred for a pin removal is neglected, the hole size on the exit side of the collimating arrangement is likely also equal to dᵣ for a given hole.

For the FC collimating arrangement, shown in FIG. 11b, the size of each hole measured on the entrance and exit surfaces are equal to a constant value, d_{F} 1115. Unlike the CC collimating arrangement shown in FIG. 11a, the entrance hole sizes in the FC collimating arrangement are generally uniform over the collimating arrangement surfaces and may be closely packed on the entrance surface 1120.

Each of the entrance holes for the TC collimating arrangement, shown in FIG. 11c has a constant size d_{T} 1120, similar to those of the FC collimating arrangement in FIG. 11b. However, the exit holes for the TC collimating arrangement are larger, and the exit hole size d_{T′} can be determined as d_{T′}=d_{T} (f+L)/L, where f is the focal length and L is the thickness of the collimating arrangement.

The sensitivity of the three exemplary collimating arrangements illustrated in FIGS. 11a-11c having different hole geometries may be compared. The penetration through the septal walls was not taken into account in this comparison. All three exemplary collimating arrangements were assumed to have the same septal thickness at the center on the entrance side. However, the septa of these focusing collimating arrangements vary in thickness with distance from the entrance surface to the exit surface, as shown in FIGS. 11a-11c. Septal thickness can also depend on the distance of a hole from the center of a CC collimating arrangement, as shown in FIGS. 10d and 11a.

The resolution of the collimating arrangement may be determined analytically by considering photon paths. Resolution can be parameterized by the full width at half-maximum (FWHM) of the point-spread function, approximated by the distance between the projections of the central ray and the most extreme ray traversing the collimating arrangement hole onto the detector. Collimating arrangement “wobbling” can be included to blur the hole pattern for a more accurate analysis, as described, e.g., in R. A. Moyer, J. Nucl. Med. 15(2):59-64 (1974).

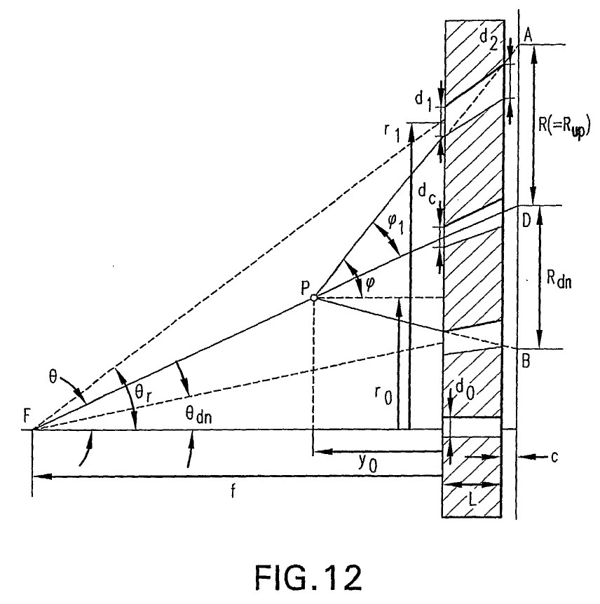

An equation provided in the Moyer publication to determined FWHM can be generalized to be applicable to any set of entry and exit hole sizes, d₁ and d₂, which may then be applied to analyze the three types of collimating arrangements shown in FIGS. 11a-11c. FIG. 12 illustrates a number of geometrical parameters that may be used to determine collimating arrangement resolution for various types of holes. Using the parameters shown in FIG. 12, an expression for the parameter R can be provided as:

$$R = \frac{( {y}_{0} + L + c )}{2 L ( f - {y}_{0} )} [ {d}_{1} ( f + L ) + {d}_{2} f ] . \qquad ( 4 )$$

Eqn. 4 can also applies to R_{dn} which is another extreme path shown in FIG. 12. The FWHM of the point spread function can be determined by dividing the value of R by the magnification factor M, where M=(f+L+c)/(f−y₀), to provide the expression:

$$F W H M = \frac{{y}_{0} + L + c}{2 L ( f + L + c )} [ {d}_{1} ( f + L ) + {d}_{2} f ] \qquad ( 5 )$$

For the TC collimating arrangement, the exit holes d₂ are larger than the entrance holes d₁ (which are equal to d_{T} 1120 in FIG. 11c). The exit hole size d₂ can be expressed in terms of f and L as

$${d}_{2} = \frac{f + L}{f} \\ {d}_{1} = \frac{f + L}{f} {d}_{T} . \qquad ( 6 )$$

The corresponding value of FWHM for the point-spread function of a TC collimating arrangement may be written as

$$F W H {M}_{TC} = \frac{( {y}_{0} + L + c ) {d}_{T}}{L} ( 1 - \frac{c}{f + L + c} ) . \qquad ( 7 )$$

For the CC and FC collimating arrangements shown in FIGS. 11a AND 11b, respectively, d₁=d₂ and the expression for FWHM can be written as

$$F W H M = \frac{( {y}_{0} + L + c ) {d}_{1}}{L} ( 1 - \frac{c + L / 2}{f + L + c} ) \qquad ( 8 )$$

Therefore, for the FC collimating arrangement, the resolution in terms of the point-spread function can be expressed as

$$F W H {M}_{FC} = \frac{( {y}_{0} + L + c ) {d}_{F}}{L} ( 1 - \frac{c + L / 2}{f + L + c} ) . \qquad ( 9 )$$

For the TC and FC collimating arrangements, the resolution may be independent of a transverse distance r₀, because they generally include a uniform hole distribution. However, for the CC collimating arrangement, d₁ may vary with r₀, and the corresponding FWHM value may also vary with respect to r₀. (or equivalently, with respect to an angle θ). The resulting expression for FWHM corresponding to the) CC collimating arrangement may be provided as

$$\begin{matrix}F W H {M}_{CC} = \frac{( {y}_{0} + L + c ) {d}_{0}}{L cos {θ}_{r}} ( 1 - \frac{c + L / 2}{f + L + c} ) , \\ where & ( 10 ) \\ cos {θ}_{r} = \frac{f}{\sqrt{{f}^{2} + {r}_{1}^{2}}} & ( 11 )\end{matrix}$$

For all collimating arrangement geometries analyzed herein, as the focal length f can become infinite (e.g., f→∞), Eqns. 8-10 are generally equivalent to the FWHM of the parallel hole collimating arrangement, which can be written as (y₀+L+c)d₀/L.

Eqn. 10 provided above, which describes the resolution of the CC collimating arrangement, may be incomplete because the central position of the extreme hole, r₁, was not provided. Based on the geometrical relationships shown in FIG. 12, this central position can be written as

$$\begin{matrix}{r}_{1} = \frac{- {f}^{2} β \pm {d}_{0} α f \sqrt{{f}^{2} + {β}^{2} - {d}_{0}^{2} {α}^{2}}}{{d}_{0}^{2} {α}^{2} - {f}^{2}} , \\ where & ( 12 ) \\ α = \frac{f}{f - {y}_{0}} ( \frac{2 {y}_{0} + L}{2 L} ) , \\ β = \frac{{fr}_{0}}{f - {y}_{0}} . & ( 13 )\end{matrix}$$

Because the CC collimating arrangement may include two extreme holes located in opposite directions from a point source P as shown in FIG. 12, there may be two distinct r₁ values. Therefore, two distinct R values, e.g. Rᵤₚ and R_{dn}, corresponding to paths PA and PB respectively, may be present. If a point source is located near the center of the CC collimating arrangement or if the collimating arrangement has a long focal length, Rᵤₚ may be approximately equal to R_{dn}. However, if r₀>>0, Rᵤₚ can be significantly larger than R_{dn}, which can yield an anisotropic point spread function. If the point spread function is anisotropic, a mean value can be obtained by averaging the FWHM value calculated for each side.

Collimating arrangement sensitivity at a particular point in space P(x₀, y₀, z₀) for a particular focal length f may be determined by calculating a fraction of photons emitted at the point P that can traverse the collimating arrangement holes. The sensitivity of the CC collimating arrangement can be determined in a closed mathematical form as described, e.g., in E. C. Frey et al., Phys. Med. Biol., 43:941-950 (1998) and in A. R. Formiconi, Phys. Med. Biol. 43:3359-3379 (1998). Such results may not apply to the collimating arrangement design described herein which includes a uniform hole size. Monte Carlo simulation programs may be used to determine the sensitivity of each collimating arrangement considered. For the TC and FC collimating arrangements, circular holes can be used or assumed. For the CC collimating arrangement, elliptical holes can be used or modeled, where these holes may have a major axis that can vary with transverse distance from the focal line.

The size of each collimating arrangement analyzed was 38 cm×52 cm. The positions of each hole on the entrance and exit surfaces were defined based on the focal length and the hole size of each collimating arrangement. 100 million gamma rays were simulated for a point source at each location. Only those which passed through both the circular or elliptical entrance holes and the corresponding exit holes were counted. Scattering in the septa was not accounted for in these analyses.

Using Eqns. 7, 9 and 10 above, the resolution of the CC, TC and FC collimating arrangements having focal lengths ranging from 20 to 50 cm can be determined. The thickness of each collimating arrangement, L, was assumed to be 3.5 cm, and the distance between the exit collimating arrangement face and the detector plane, c, was selected to be 4 mm. A septal thickness t at the entrance surface of 0.16 mm was assumed for all collimating arrangements analyzed, and the septal penetration and scattering effects were not considered. Because the resolution can vary with both axial distance (along the focal line, y₀) and transverse distance from the center r₀, FWHM values were averaged at five transverse positions with an axial distance of 10 cm from the surface of the collimating arrangement. The transverse positions used were r=0, 2, 4, 6, and 8 cm from the focal line.

FWHM values were calculated for the CC collimating arrangement for focal distances ranging from 20-50 cm. For each focal distance, hole sizes which would yield the same value of FWHM in the FC collimating arrangement and the TC collimating arrangement were determined. These results are summarized in Table 1.

A sensitivity was determined as counts per 10,000 photons emitted from a point source at various locations using the hole sizes shown in Table 1. The sensitivity of the three types of cone-beam collimating arrangements at a point on the focal line 10 cm from the collimating arrangement surface are provided as a function of focal length f in FIG. 13. The differences in the sensitivity among the collimating arrangements were observed to be larger for shorter focal lengths. For example, the sensitivity gain for a TC collimating arrangement, compared to that of the CC collimating arrangement, was observed to be 7% and 45% for focal lengths of 50 cm and 20 cm, respectively. For an FC collimating arrangement, the sensitivity gain was observed to be 1.5% and 25% for focal lengths of 50 cm and 20 cm, respectively.

TABLE 1

| f (cm) | FWHM (cm) | d₀ (cm) | d_{F} (cm) | d_{T} (cm) |
|---|---|---|---|---|
| 20 | 0.642 | 0.16 | 0.1777 | 0.1645 |
| 30 | 0.613 | 0.16 | 0.1647 | 0.1561 |
| 40 | 0.612 | 0.16 | 0.1621 | 0.1556 |
| 50 | 0.615 | 0.16 | 0.1612 | 0.1559 |

FWHM values for CC collimating arrangements having hole diameter d₀ = 0.16 cm and the indicated focal lengths f, together with hole diameters d_{F} and d_{T} for FC and TC collimating arrangements, respectively, needed to yield corresponding FWHM values.

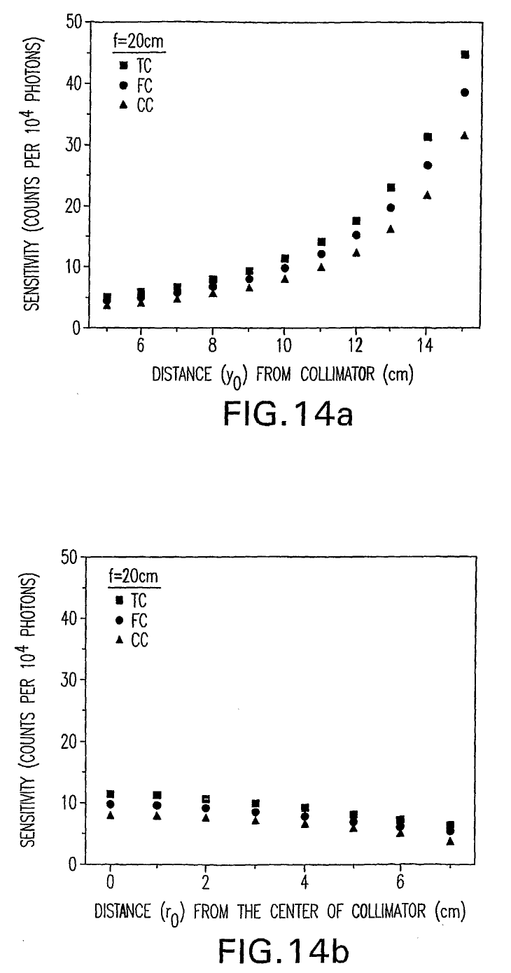

The sensitivity of the three types of the collimating arrangements (CC, FC and TC), each having a focal length of 20 cm, were determined for various point source locations. FIG. 14a shows a graph of exemplary sensitivity data as a function of point source distance in a direction perpendicular to the surface of each collimating arrangement. The sensitivity was determined along the focal line for distances ranging from about 5 m to 15 cm. The collimating arrangement sensitivity was observed to increase with increasing distance due to increasing magnification. The sensitivity of the TC collimating arrangement, averaged over the various distances analyzed, was observed to be about 44% greater than that of the CC collimating arrangement. The average gain of the FC collimating arrangement compared to the CC collimating arrangement was about 23%.

FIG. 14b shows a graph of exemplary variations in the collimating arrangement sensitivity as a point source is moved in a transverse direction from the center of focal line (at y₀=10 cm). Sensitivity was observed to decrease with increasing distance between the point source and the focal line. The average gains for the TC and FC collimating arrangements compared to that of the CC collimating arrangement for transverse variations in the point source location were approximately the same as the gains observed for variations in the perpendicular direction, e.g., about 44% and 23% respectively.

For example, the sensitivity of the collimating arrangements having different hole patterns (i.e., CC, FC and TC) was compared at equal resolution values for focal lengths ranging from about 20 cm, a value that may be useful for the USCB collimating arrangement, to about 50 cm, a value typical of cone-beam collimating arrangements that may be used in a clinical setting. It was observed that both the FC and TC collimating arrangements exhibited improved sensitivity as compared to a standard CC collimating arrangement configuration. The sensitivity of these collimating arrangements, each having a focal length of about 20 cm, were evaluated for various locations of a point source. For all locations analyzed the TC collimating arrangement exhibited the highest sensitivity, with an overall gain of about 44% as compared to the conventional CC collimating arrangement. The gain of the FC collimating arrangement was about 23% as compared to the CC collimating arrangement.

As described herein above, the USCB collimating arrangement may be provided on a dual-head SPECT system together with a fan-beam collimating arrangement for data sufficiency. This configuration can yield improved sensitivity throughout the imaging volume as compared to a similar apparatus provided with parallel-hole collimating arrangements. The sensitivity provided by the USCB collimating arrangement used together with a fan-beam collimating arrangement may increase by a factor ranging from about 10 to 30 using a conventional USCB collimating arrangement manufactured by a casting technique. The TC and FC collimating arrangements described herein have different hole configurations and can be manufactured by other techniques such as, e.g., photoetching and SL. These exemplary USCB collimating arrangement designs can provide even greater sensitivity gains resulting from close packing of peripheral holes and tapering of holes.

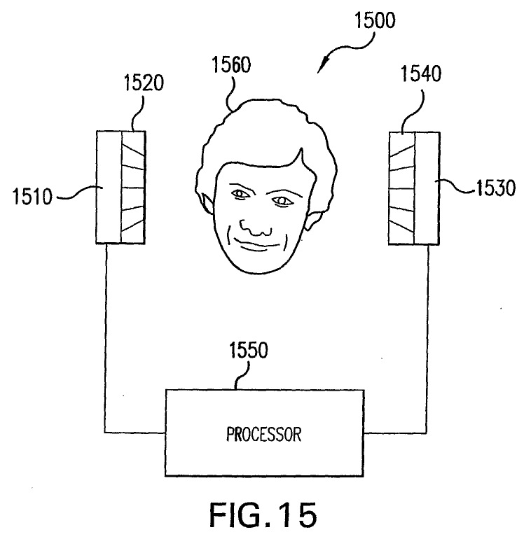

An exemplary diagram of a system in accordance with an exemplary embodiment of the present invention is shown in FIG. 15. This system can be configured to generate imaging data for a patient's head 1560 or another body organ. For example, this exemplary system 1500 can include a cone-beam collimating arrangement 1520 associated with a first detector 1510, and a fan-beam collimating arrangement 1540 associated with a second detector 1530. The detectors 1510, 1530 can be configured to detect a radiation emanating from the head 1560 which passes through the collimating arrangements 1520, 1540. Such radiation may be generated by certain substances, such as radionuclides, which can be ingested or otherwise introduced into a patient. The detectors 1510, 1530 can be configured to communicate with a processing arrangement 1550, which may include a computer. The processing arrangement 1550 can be configured to generate image data based on signals received from the detectors 1510, 1530. The detectors 1510, 1530 and associated collimating arrangements 1520, 1540 can be mounted to a structure (not shown in FIG. 15) which can allow the detectors 1510, 1530 to be rotated around the head 1560 in specified increments. In this manner, the system 1500 can be used to collect data when the detectors 1510, 1530 are in a variety of positions relative to the head 1560. Signals collected from a number of particular positions of the detectors 1510, 1530 can be used to generate image data which in turn may be used to generate three-dimensional images and/or two-dimensional cross-sections.

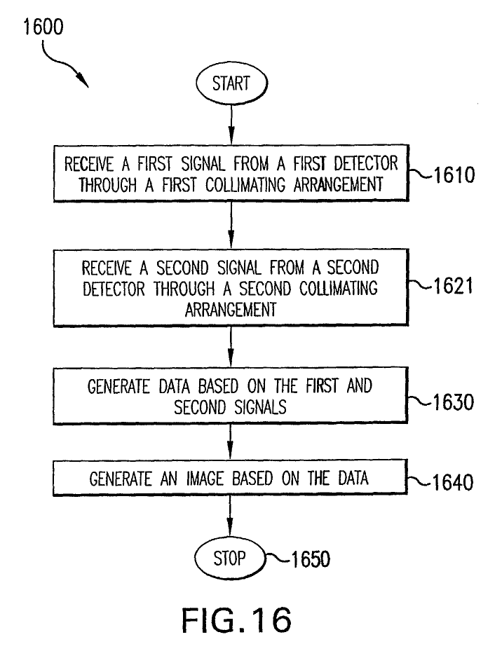

An exemplary flow diagram 1600 of an exemplary method in accordance with certain embodiments of the present invention is shown in FIG. 16. A first signal can be received from a first detector through a first collimating arrangement (step 1610). The signal can be, e.g., a radiation emitted from an object to be imaged such as, e.g., a human brain. The first collimating arrangement can be a cone-beam collimating arrangement such as those described herein above, which can have a focal point located within the brain or other organ being imaged. A second signal can be received from a second detector through a second collimating arrangement (step 1620). The second collimating arrangement can be a fan-beam collimating arrangement such as those described herein above, which can have a focal length selected, e.g., such that the entire brain or other organ being imaged lies within its field of view. The positions of the detectors may be shifted relative to the object being imaged by specified amounts and further signals can be obtained in the new position. This procedure can be repeated until signals from a sufficient number of positions are obtained. These signals may then be provided to a processing arrangement such as, e.g., a computer, and used to generate data associated with an image of the object (step 1630). The data may then be used to generate images of the object (step 1640). The images may be, e.g., three-dimensional images and/or two-dimensional cross-sections of the object. After images are generated, the procedure may be stopped (step 1650).

Although only certain exemplary embodiments of this invention have been described in detail above, those skilled in the art will readily appreciate that many modifications are possible in the exemplary embodiments without departing from the novel teachings and advantages of this invention. Accordingly, all such modifications are intended to be included within the scope of this invention.

For example, exemplary embodiments of the present invention are described herein above that can include a cone-beam collimating arrangement having a focal length of about 20 cm, and a fan-beam collimating arrangement having a focal length of about 40 cm. These focal lengths represent typical values that may be appropriate for imaging an average human brain using SPECT techniques. In general, the focal length of the cone-beam collimating arrangement can be selected such that the focal point lies within the brain or other organ being imaged when the collimating arrangement is mounted to the SPECT system or similar detector apparatus. Appropriate values for a focal length of a cone-beam collimating arrangement can be, e.g., between about 17 and 23 cm, and/or between about 19 and 21 cm. The particular focal length selected for a given application can depend at least in part on the size of the object being imaged.

The focal length of a fan-beam collimating arrangement that may be used in accordance with certain exemplary embodiments of the present invention may also be varied depending on the characteristics of the object being imaged. Exemplary fan-beam collimating arrangements having a focal length of about 40 cm are described herein in detail. In general, the focal length should be selected to be large enough such that most or all of the organ (e.g., a brain) being imaged is within the field of view of the fan-beam collimating arrangement. This exemplary criterion can assist in to ensuring that sufficient data is obtained to provide an improved image quality and prevent cut-off of portions of the image obtained. A focal length less than 40 cm can be selected, e.g. about 35 cm or 36-38 cm, if a smaller organ is being imaged. A focal length of about 40 cm, or between about 40 and 45 cm, or up to about 50 cm, may be appropriate for imaging a typical human brain. A much larger focal length, e.g., a focal length greater than about 50 cm, may not be desirable because the performance of the fan-beam collimating arrangement may begin to approach that of a parallel-hole collimating arrangement for very large focal lengths.

In further embodiments of the present invention, a triple-head SPECT apparatus may be used with three collimating arrangements. At least one collimating arrangement can be a fan-beam collimating arrangement to ensure data sufficiency. Two cone-beam collimating arrangements may be used together with the fan-beam collimating arrangement, which may yield more detailed images. For example, two cone-beam collimating arrangements having different focal lengths and/or hole geometries may be used to provide improved image detail. In this exemplary configuration, the focal point of each cone-beam collimating arrangement can be selected to lie within the organ being imaged during the imaging procedure.

The thickness and hole size selected for each collimating arrangement used in accordance with exemplary embodiments of the present invention can be selected based at least in part on the specific radionuclides or other detected substances used. Conventional criteria may be used when selecting these collimating arrangement parameters.

Exemplary embodiments of the present invention may be used to image a variety of organs, in addition to the brain as described in detail herein. For example, procedures such as cardiac imaging or pediatric imaging may be performed, as well as imaging of other organs or organisms that can be examined using SPECT techniques in conjunction with the exemplary embodiments of the present invention.

It should further be noted that any patents, applications and publications cited herein above are incorporated herein by reference in their entireties.

1. An apparatus for providing at least one image of at least one portion of a bodily organ, comprising:
a first detecting arrangement and a second detecting arrangement;
a first collimating arrangement associated with the first detecting arrangement, wherein the first collimating arrangement comprises a cone-beam collimating arrangement and wherein a focal point of the first collimating arrangement is provided within the at least one portion of the bodily organ; and
a second collimating arrangement associated with the second detecting arrangement, wherein the second collimating arrangement comprises a fan-beam collimating arrangement and wherein all of the at least one portion of the bodily organ is provided within a field of view of the second collimating arrangement.

2. The apparatus according to claim 1, wherein the cone-beam collimating arrangement has a focal length between about 17 cm and 23 cm.

3. The apparatus according to claim 1, wherein the cone-beam collimating arrangement has a focal length between about 19 cm and 21 cm.

4. The apparatus according to claim 1, wherein the cone-beam collimating arrangement has a focal length of about 20 cm.

5. The apparatus according to claim 1, wherein the fan-beam collimating arrangement has a focal length between about 35 and 40 cm.

6. The apparatus according to claim 1, wherein the fan-beam collimating arrangement has a focal length between about 36 and 38 cm.

7. The apparatus according to claim 1, wherein the fan-beam collimating arrangement has a focal length between about 40 and 50 cm.

8. The apparatus according to claim 1, wherein the fan-beam collimating arrangement has a focal length between about 40 and 45 cm.

9. The apparatus according to claim 1, wherein the fan-beam collimating arrangement has a focal length of about 40 cm.

10. The apparatus according to claim 1, wherein the cone-beam collimating arrangement comprises holes having an approximately constant size throughout the cone-beam collimating arrangement.

11. The apparatus according to claim 1, wherein the cone-beam collimating arrangement comprises holes having a constant size over a front surface of the cone-beam collimating arrangement, and wherein a cross-section of at least a portion of the holes increases with depth in the cone-beam collimating arrangement.

12. The apparatus according to claim 1, wherein the cone-beam collimating arrangement comprises a hybrid ultra-short cone-beam/slant collimating arrangement.

13. The apparatus according to claim 1, further comprising a third detecting arrangement and a third collimating arrangement associated with the third detecting arrangement, wherein the third collimating arrangement comprises a further cone-beam collimating arrangement, and wherein a focal point of the third collimating arrangement is provided within the at least one portion of the bodily organ.

14. The apparatus according to claim 13, wherein a focal length of the first collimating arrangement is different from a focal length of the third collimating arrangement.

15. The apparatus according to claim 1, wherein the bodily organ is a brain.

16. The apparatus according to claim 1, wherein the bodily organ is a heart.

17. A method for generating data associated with an image of at least one portion of a bodily organ, comprising:
receiving a first signal from a first detecting arrangement, wherein the first signal is associated with first radiation emitted from the at least one portion of the bodily organ that passes through a first collimating arrangement which has a focal point within the at least one portion;
receiving a second signal from a second detecting arrangement, wherein the second signal is associated with second radiation emitted from the at least one portion of the bodily organ that passes through a second collimating arrangement, and wherein all of the at least one portion of the bodily organ lies within a field of view of the second collimating arrangement; and
generating the data based on the first signal and the second signal.

18. The method of claim 17, further comprising generating at least one image based on the data.

19. A software arrangement for generating image data associated with at least one portion of a bodily organ, comprising:
a first set of instructions which, when executed by a processing arrangement, is capable of receiving a first data set from a first detecting arrangement, wherein the first data set is associated with a position of the first detector and with first radiation emitted from the at least one portion of the bodily organ that passes through a first collimating arrangement having a focal point within the at least one portion of the bodily organ;
a second set of instructions which, when executed by the processing arrangement, is capable of receiving a second data set from a second detecting arrangement, wherein the second data set is associated with a position of the second detector and with second radiation emitted from the at least one portion of the bodily organ that passes through a second collimating arrangement, and wherein all of the at least one portion of the bodily organ lies within a field of view of the second collimating arrangement; and
a third set of instructions which, when executed by the processing arrangement, is capable of generating the image data based on the first data set and the second data set.
